(19) United States
(12) Patent Application Publication
(10) Pub. No.: US 2009/0269798 A1
(43) Pub. Date: Oct. 29, 2009

(54) METHOD AND SYSTEM FOR SELECTING CHEMOTHERAPEUTIC AGENTS

(75) Inventors: Lisa A. Beltz, Cedar Falls, IA (US); John A. Bumpus, Cedar Falls, IA (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: UNIVERSITY OF NORTHERN IOWA RESEARCH FOUNDATION, Cedar Falls, IA (US)

(21) Appl. No.: 12/429,410

(22) Filed: Apr. 24, 2009

Related U.S. Application Data
(60) Provisional application No. 61/047,639, filed on Apr. 24, 2008.

(51) Int. Cl. C12Q 1/02

(52) U.S. Cl. 435/29

(57) ABSTRACT

Described herein is a method and corresponding system, including reagents and assays, for determining the in vivo efficacy of drugs in the course of chemotherapy. The method can include identifying a patient having a condition, such as leukemia, that is potentially susceptible to one or more candidate drugs having a particular in vivo biochemical activity, such as the generation of reactive oxygen or nitrogen species. An in vitro biochemical assay is provided that corresponds to the biochemical activity, and a tissue sample is tested to determine the responsiveness of the tissue sample to the biochemical activity, for instance, the ability of the tissue sample to degrade or inhibit the active species (in this case, hydrogen peroxide), and thereby prevent its desired effect against the cancerous cells. A course of chemotherapy can be based, at least in part, on the responsiveness of the tissue sample to the biochemical activity.

### TECHNICAL FIELD

The invention relates to the field of chemotherapy, including methods and systems for the selection and use of agents having desired efficacy. In another aspect, the invention relates to methods for evaluating the usefulness of agents that function by means of a pathway that includes reactive oxygen or nitrogen species.

### BACKGROUND OF THE INVENTION

Typical methods for determining a drug therapy regimen have long involved a process of trial and error, in which chemotherapeutic agents and corresponding dosages are initially determined based upon various indications such as physical symptoms, patient size, sex and age, clinical indications (e.g., test results), history, and other medical conditions, including potential allergies. Based upon these various factors, the physician typically prescribes one or more agents, and then monitors the effectiveness of these agents over time, in order to change either agents and/or dosages as may be needed.

In certain areas, tests have been developed to help determine a treatment based on the actual or predicted susceptibility of the patient to particular agents. For instance, antimicrobial susceptibility testing is commonly used to determine the likelihood of successfully treating a patient's infection with a particular antimicrobial agent. Antimicrobial susceptibility (sensitivity) testing is often performed for aerobic isolates considered as significant pathogens, though antimicrobial susceptibility testing of anaerobes is not performed as often.

Similarly, over recent years, a field has emerged that is known alternatively as “theranostics” or at times “personalized” medicine, which generally involves determining the patient's own genetics or other biomarkers in order to then design a suitable treatment strategy.

Unfortunately, for many agents, including many that have been recently developed and are in widest use, agent selection and corresponding dosages continue to be determined by conventional trial and error. For instance, the leukemia drug daunorubicin belongs to the general group of medicines known as antineoplastics used to treat some kinds of cancer. Daunorubicin interferes with the growth of cancer cells, which are eventually destroyed. Structurally, this drug falls within the category of drugs known as the anthracyclines. The anthracyclines include drugs that are antibiotics as well as drugs that are chemotherapeutic agents. They function by intercalating into the base pairs in the minor grooves in DNA. They also cause free radical mediated damage of deoxyribose in DNA. Free radicals are reactive and potentially deleterious molecules that possess one or more unpaired electrons. Daunorubicin mediates formation of superoxide cation radical, hydroxyl radical and hydrogen peroxide by redox cycling. Two other anthracycline chemotherapeutic drugs that are effective against some forms of cancer are doxorubicin and idarubicin.

Currently, before treating with daunorubicin, the patient and doctor are advised to talk about both the drug's benefits and risks, at which time they should consider various factors, such as allergies, pregnancy, breast-feeding, age (including corresponding concerns such as heart problems), other medical problems, and other medicines the patient may be taking that might affect the use of daunorubicin. In turn, the dose of daunorubicin will be different for different patients. Both the selection and dose of any such drug may depend on a number of things, including what the medicine is being used for, the patient's size, and whether or not other medicines are also being taken.

A handful of references appear to address susceptibility to drugs such as daunorubicin, though generally relate to postulated and/or complex methods such as genetic testing, that clearly require significant time, cost, and technical capabilities, and appear to not be in common clinical use.

### SUMMARY OF THE INVENTION

The present invention provides a method and corresponding system, including components such as reagents and assays for use in such system, for determining the in vivo efficacy of agents in the course of chemotherapy, the method comprising the steps of:
1) identifying a patient having a condition potentially susceptible to one or more candidate agents having a particular in vivo biochemical activity;
2) providing an in vitro biochemical assay corresponding to the biochemical activity;
3) obtaining a suitable sample from the patient;
4) performing the biochemical assay on the tissue sample under conditions suitable to determine the responsiveness of the tissue sample to the biochemical activity,
5) determining a course of chemotherapy based, at least in part, on the responsiveness of the tissue sample to the biochemical activity.

In a preferred embodiment,
a) the in vivo biochemical activity of the candidate agents comprises the direct or indirect generation of either reactive oxygen species and/or reactive nitrogen species (e.g., drugs having ROS or RNS mediated activity);
b) the biochemical assay comprises means for exposing a patient sample in vitro to reactive oxygen species and/or reactive nitrogen species; and
c) the responsiveness of the tissue sample to the biochemical activity comprises the ability of the tissue sample to inactivate the corresponding reactive species (for instance, identifying cells or samples having endogenous antioxidant or antinitrosant activity).

In turn, Applicant has determined the manner in which the ability of a tissue (e.g., cancer tissue) sample to inactivate the biochemical effect of the drug in vitro, can be used to determine the efficacy of the agent in vivo.

In one preferred embodiment, the patient condition is leukemia, and the candidate drugs are those that function by means of the generation of reactive oxygen species. A preferred assay of this invention can be designed to determine whether an individual's leukemia cells are likely to be treatable by chemotherapeutic drugs that act via generation of reactive oxygen species (ROS), particularly hydrogen peroxide.

In such an embodiment, the method and assay of this invention can be used to determine whether or not an individual patient's leukemia cells are susceptible to the ROS or RNS classes of anti-leukemic drugs, by determining whether a tissue sample comprising the cells is able to degrade or inhibit the active species in vitro. The method can also help to determine the relative amount of these drugs to use initially in treating an individual patient. This can save valuable time if a high drug dose is recommended, and alternatively, can spare the patient unnecessary, often severe, side-effects if lower doses can be reasonably employed. In situations in which a patient was to be tested at regular intervals during his/her treatment, the assay can also be used to detect changes occurring in the remaining leukemic cell populations within the patient. If a patient responded well to the ROS- or RNS-generating category of drugs initially, the bulk of the leukemic cells are likely to be readily killed. This can allow for the outgrowth of a minor population of leukemic cells which is more resistant to these drugs. Regular monitoring of a small sample of the patient's blood can detect the presence of such resistant cells early and allow drugs functioning by a different mechanism to be added to the treatment regimen in order to kill these cells before they are able to gain a strong foothold in the patient.

In one preferred embodiment, the method of this invention can be provided in the form of a simple, relatively inexpensive clinical assay which requires only a small tissue sample (e.g., peripheral blood, bone marrow, solid tumor or tissue, or cerebral fluid), and which in turn, can determine within a short period of time the potential efficacy of any of an entire class of drugs.

The method of this invention is particularly amenable for use in a clinical chemistry/laboratory medicine reference laboratory, which currently tend to provide hundreds or more assays for use in diagnosis and treatment. For instance, various clinical laboratories tend to include an interdisciplinary group of laboratorians, geneticists, and physicians that provide biochemical testing and result interpretation for the diagnosis, study, and clinical care of patients, for instance, those with inborn errors of metabolism, malabsorption, and malnutrition disorders. One or more assays of the present invention would provide an ideal fit and opportunity for such reference laboratories to expand their offerings, and in turn, benefit patients and medical practitioners alike.

In one preferred embodiment, the condition is a cancer such as leukemia, and the candidate drugs are those that function by means of the direct or indirect generation of reactive oxygen species or reactive nitrogen species that are cytotoxic for the cancer cells. A preferred assay of this invention can be designed to determine whether an individual's leukemia cells are likely to be killed by such drugs, by determining whether the cells, or a sample in which they exist, are capable of degrading superoxide or hydrogen peroxide and thus preventing production of even more reactive (and more toxic) ROS or RNS before the drugs can have the desired action.

In other optional embodiments, the method of this invention can be used, or adapted for use, in testing normal tissues for susceptibility to ROS or RNS, thereby serving as a tool to predict possible side effects (e.g., leukopenia).

Among other aspects, Applicant has shown that the dose dependent enzyme mediated rate of removal or degradation of hydrogen peroxide (which is itself an ROS) is different in leukemia cells than it is in normal cells. This is important because hydrogen peroxide is a co-substrate required for the subsequent formation of other, more harmful, reactive oxygen species and reactive nitrogen species (ROS and RNS). Based upon this, the hydrogen peroxide dependent differential killing of normal and leukemia cells that the Applicant has observed is believed to be enzyme dependent, at least in part, with catalases and peroxidases appearing to be key enzymes responsible for the enzyme-catalyzed removal/degradation of hydrogen peroxide, thereby corresponding with either the survival or killing of the cells themselves. Similar assays might be performed to study cellular removal of superoxide by superoxide dismutase within the cells.

In turn, a method of this invention can include any means for determining the relative and/or absolute levels of such enzymes in a sample, in either quantitative or qualitative terms, including by means of a bioassay as described herein, or by means of chemical or biochemical assays (SDS-polyacrylamide gel electrophoresis or high performance liquid chromatography, for example), using techniques that will become apparent to those skilled in the art, when given the present description. The enzyme levels can be determined in different samples or within different portions (e.g., different cell types) within a single sample, such as within normal as opposed to cancerous cells.

### DETAILED DESCRIPTION

Reactive oxygen species (ROS) that can act as an oxidant in the course of drug therapy can include, for instance, hydroxyl radicals, hydrogen peroxide, and singlet oxygen. Reactive nitrogen species (RNS) that can act as a nitrosant can include nitric oxide, peroxynitrite, and nitrogen dioxide.

In turn, a wide variety of drugs have activity that includes the direct or indirect production of ROS or RNS in vivo, thereby providing oxidative or nitrosative activity, respectively.

Such drugs can lead to cell death in a variety of ways, e.g., the activation of cell signaling pathways to include MAPK/ERK kinase (MEK), and extracellular signal-regulated kinase (ERK) phosphorylation, by increased expression of inflammatory cytokines (e.g., tumor necrosis factor alpha [TNFalpha], interleukin-1 [IL-1]), and by activation of specific transcription factors (e.g., NF kappaB, AP-1). Moreover, such drugs can initiate apoptosis in response to oxygen- and nitrogen-based free radicals, leading to mitochondrial dysfunction, increased gene expression of death receptors, and/or their ligands (TNFalpha, Fas ligand [FasL]).

Applicant has discovered the manner in which simple, conventional assays for determining ROS or RNS inactivation in vitro can be adapted for use in the method of the present invention, given the teaching of this invention.

The present invention provides a method and corresponding system, including components such as reagents and assays for use in such system, for determining the in vivo efficacy of drugs in the course of chemotherapy, the method comprising the step of identifying a patient having a condition potentially susceptible to one or more candidate drugs having a particular in vivo biochemical activity.

In one preferred embodiment, the condition is a cancer such as leukemia and the candidate drugs are selected from the group consisting of daunorubicin, doxorubicin, cytarabine, parthenolide, mitomycin C, adriamycin, carminomycin, 7-0-methylnogalarol, aclacinomycin A, benzanthraquinones, streptonigrin, and cisplatin, as well as derivatives and analogues thereof.

Daunorubicin is often used in the treatment of acute myelogenous leukemia (AML), acute lymphocytic leukemia (ALL), chronic myelogenous leukemia (CML)—blast crisis, neuroblastoma. See: Biochemistry Journal, 1981. 194:369-72. Doxorubicin is often used in the treatment of AML, ALL, Hodgkin's lymphoma (HL), Non-Hodgkin's lymphoma (NHL), neuroblastoma. See: Biochemistry Journal, 2005. 381:527-539 and Proceedings of the National Academy of Science USA, 2004. 101:13227-13232. Cytarabine (Ara-C) is used in the treatment of NHL and other leukemias, while Parthenolide (PTL) is used in the treatment of AML, CML—blast crisis. See: Journal of Cellular Investigation, 1998. 102:1961-1968.

Other chemotherapeutic agents which act via ROS generation: mitomycin C, adriamycin, carminomycin, 7-0-methylnogalarol, aclacinomycin A, benzanthraquinones, streptonigrin (Proceedings of the National Academy of Science USA, 1979. 76:954-957), cisplatin (used to treat metastatic testicular, ovarian, advanced bladder carcinoma and cancers of the head and neck, lungs, and cervical cancers).

The method further involves the step of providing an in vitro biochemical assay corresponding to the biochemical activity. Given the present description, those skilled in the art of performing biochemical assays (e.g., determining enzyme activity and product formation) will appreciate the manner in which an assay can be developed and implemented for use in clinical settings.

In a particularly preferred embodiment, the in vivo drug activity includes the hydrogen peroxide dependent generation of ROS or RNS, and in turn, the in vitro assay can be based upon the ability of the tissue sample to degrade hydrogen peroxide that is provided or generated in vitro, thus preventing generation of other reactive oxygen and reactive nitrogen species (ROS and RNS) thereby predicting the extent to which the drug might be efficacious in vivo.

In turn, the method involves the step of obtaining a suitable tissue sample from the patient. Such a sample can be obtained in any suitable manner and from any suitable source, e.g. from diseased tissue per se, or from other, non-diseased tissue that may be directly or indirectly related to both drug and condition (e.g., ability of a gland to be treated so as to increase or decrease production of an agent having role in cancer growth)

In turn, the biochemical assay is performed on the tissue sample under conditions suitable to determine the responsiveness of the tissue sample to the biochemical activity. For instance, hydrogen peroxide is commercially available and can be added directly to the tissue or cell sample. Those skilled in the art, given the present description, will be able to determine a suitable manner for providing any desired ROS or RNS under the conditions of use, e.g., a xanthine oxidase based generating system can be used to produce superoxide.

In a preferred embodiment, the assay is an automated one that can be run promptly and efficiently within the large scale operation of a conventional laboratory medicine reference laboratory. A standard hydrogen peroxide assay can be accomplished, for instance, within about 1 to 1½ hours following cell separation. A standard superoxide based assay can be accomplished within about 2 to 2½ hours. The assays of this invention lend themselves well to automation, e.g., the use of a 96-well plate and corresponding systems for handling and reading such plates using robotic technology.

Finally, the results of the biochemical assay can be used to determine a course of chemotherapy based, at least in part, on the responsiveness of the tissue sample to the biochemical activity.

Individuals having leukemia can vary greatly in their responsiveness to any given chemotherapeutic drug since each person's leukemia is a result of a unique combination of mutations occurring within that person. In turn, Applicant has found that just as different leukemic cell lines, originating from different individuals, differ in their susceptibility to damage by hydrogen peroxide, which in turn relates to their ability to eliminate this ROS, so to are leukemic cells from different individuals also likely to differ in these properties. Applicant has discovered that these differences can be easily and quickly tested by determining rate of removal of hydrogen peroxide from cell cultures, in a manner that does not require expensive antibodies, DNA assays, or use of radioactive compounds. In turn, an assay of this invention can be used to reliably predict the extent to which an individual's leukemia cells will respond to those chemotherapeutic agents which act via superoxide or hydrogen peroxide generation. The assay tends to be quite reproducible as well, e.g., with standard deviation of less than about 2%.

For instance, Applicant has found that normal leukocytes and cells of one type of leukemic human Jurkat T lymphoblastic leukemia) differ greatly in their susceptibility to damage by hydrogen peroxide. In turn, these cells also had great differences in their ability to remove hydrogen peroxide from the cultures. When additional types of leukemic cells were tested, it was found that those cells which remove hydrogen peroxide from the cultures quickly were not readily damaged by this ROS. In turn, many currently used anti-leukemia drugs function by generating hydrogen peroxide which then damages cellular structures, such as membrane lipids, proteins, and DNA, either directly or by interacting with other molecules within the cell to form even more toxic compounds.

Before a particular biochemical assay is suitable for clinical use, various types of experiments are typically performed. For instance, the cell lines to be used will generally need to be tested for differential susceptibility to either growth suppression or killing by 2-4 different chemotherapeutic drugs that function via generation of ROS.

In addition, samples of leukemia cells taken directly from patients prior to chemotherapy with a ROS-generating drug regimen can be obtained and tested for their relative abilities to eliminate hydrogen peroxide from cultures and, further, for their susceptibility to growth suppression or killing by hydrogen peroxide in vitro. Using relative hydrogen peroxide-climination activity as a criterion, it will be possible to predict responsiveness of each patient's cells to hydrogen peroxide in vitro and the drug regimen in vivo.

This invention can be readily adapted for use in screening susceptibility to this same category of ROS-generating drugs of solid tumors of virtually any origin, providing that a tumor biopsy sample were made available. This would involve a more invasive technique than the blood sample required in the described leukemia assay.

Production of ROS from molecular oxygen occurs in the blood via oxidation of hemoglobin in the red blood cells and by several types of leukocytes (polymorphonuclear neutrophils, monocyte/macrophages). ROS are highly reactive, destructive, oxidating agents. They damage microbial/cellular proteins, cell membranes (via lipid peroxydation), and mutate DNA. These activities play vital roles in killing harmful agents, such as microbes and cancerous cells. Reactive oxygen species frequently cause a number of serious human diseases, including cancer and neurodegenerative disorders. They may also be a major factor in cellular damage that results in aging. Cells, especially those that produce ROS, contain a variety of protective enzymes that convert these molecules into less harmful compounds. Different cell types contain different protective enzymes and have differing levels of these enzymes. Due to the deleterious effects of ROS, many health-care professions are now urging people to include foods/beverages/supplements with known anti-oxidant activity into their diets.

An assay of this invention can be used for other purposes as well. For instance, while ROS provide a valuable tool by which the immune system kills microbes and cancerous cells, ROS are also responsible for much of the tissue damage occurring in the human body which results in tissue/organ death and/or disease. If cells from different individuals vary in their ability to eliminate the hydrogen peroxide, produced by that individual's own immune cells or derived from ingested foods, to which they are inevitably exposed, different individuals may have differing degrees of susceptibility to these diseases. Cells from an individual who is known to be genetically predisposed to developing such an ROS-induced disease can be tested in order to determine the relative susceptibility of their cells to various types of ROS based upon the cells' ability to eliminate hydrogen peroxide. Such a person's leukocytes can be tested in order to determine whether they produce a higher than normal amount of hydrogen peroxide. Cells from the organ of concern (such as the liver) can be tested for their relative ability to eliminate hydrogen peroxide. Leukocytes can be readily obtained from a small blood sample, while cells from other organs would require the more invasive biopsy. If the individual's leukocytes produced large amounts of hydrogen peroxide or the cells from their organs were found to eliminate it poorly, that individual can be advised to switch to a diet that contains high amounts of foods or dietary supplements known to contain anti-oxidants (green or red tea, blueberries, vitamin C).

The noninvasive testing of blood samples can be used to determine if a person generates higher than normal levels of ROS, and can also be expanded to include normal individuals. Since many neurodegenerative diseases (Alzheimer's, Parkinson's, and Huntington's diseases, ALS, and age-associated memory loss) are in a large part due to neural exposure to ROS, such information can be useful in suggesting dietary modifications that can decrease the risk of developing such diseases in high ROS producing persons. This is of particular importance at this time since an increasing proportion of the population is living to ages in which these diseases are common.

### EXAMPLES

In the experiments shown in Table1 a/b, the indicated concentrations of hydrogen peroxide were added to the various types of cells and they were incubated for 96 hours until growth was measured by the ³H-thymidine assay or viability determined by the 3-(4,5-diphenylthiazol-2-yl)-2,5-diphenyltetrazolium bromide (“MTT”) assay. Numbers represent triplicate or quadruplicate determinations.

TABLE 1a
Growth After Exposure to Hydrogen Peroxide - Five Cell Types

| | Hydrogen Peroxide Added to Cells (μM) | | | |
|---|---|---|---|---|
| Cell Types | 25^{a} | 50 | 100 | 200 |
| Normal cells | 68.2 ± 5.4^{b} | 84.4 ± 12.3 | 92.5 ± 19.9 | 81.9 ± 9.2 |
| THP-1 | 115.8 ± 10.6 | 102.8 ± 5.9 | 91.0 ± 0.2 | 63.2 ± 7.1 |
| YT-INDY | 109.2 ± 2.4 | 2.8 ± 0.8 | 3.7 ± 2.5 | 10.2 ± 3.5 |
| K-562 | 41.6 ± 24.5 | 7.1 ± 5.1 | 4.3 ± 2.6 | 2.7 ± 0.3 |
| Jurkat | 1.2 ± 0.4 | 2.4 ± 1.1 | 1.3 ± 1.1 | 1.0 ± 0.2 |

^{a}Amount of hydrogen peroxide added to the cells.
^{b}Data indicates the amount of cell division (compared to control cells without hydrogen peroxide) remaining after exposure to hydrogen peroxide for 96 hours ± standard deviation. The lower the number, the greater the growth suppression by hydrogen peroxide.
Cell types:
THP-1 = human monocytic leukemia
YT-INDY = human NK cell leukemia
K-562 = human erythroleukemia
Jurkat = human T lymphocytic leukemia

The data in Table 1a indicates that the growth of normal cells and THP-1 cells was minimally affected by hydrogen peroxide. YT-INDY growth was affected to a greater extent, that of K-562 more, and that of Jurkat cells was suppressed to the greatest extent.

TABLE 1b
Viability After Exposure to Hydrogen Peroxide

| | Hydrogen Peroxide (μM) Added to Cells | | | |
|---|---|---|---|---|
| Cell Types | 25^{a} | 50 | 100 | 200 |
| Normal cells | 100.6 ± 3.3^{b} | 102.7 ± 5.5 | 89.4 ± 0.9 | 83.0 ± 1.8 |
| THP-1 | 103.0 ± 2.2 | 101.8 ± 1.8 | 95.6 ± 0.6 | 65.6 ± 2.0 |
| YT-INDY | 68.2 ± 4.8 | 32.1 ± 2.6 | 29.7 ± 1.0 | 27.5 ± 0.5 |
| K-562 | 58.6 ± 3.2 | 19.6 ± 1.2 | 8.9 ± 0.2 | 8.0 ± 0.4 |

^{a}Amount of hydrogen peroxide added to the cells.
^{b}Data indicates the level of live cells (compared to control cells without hydrogen peroxide) remaining after exposure to hydrogen peroxide for 96 hours ± standard deviation. The lower the number, the greater the numbers of cells killed by hydrogen peroxide.

The data in Table 1b indicates that the minimal killing of normal cells and THP-1 cells occurred after their exposure to hydrogen peroxide. YT-INDY was killed to a greater extent and that of K-562 more. It was also found that significant killing of Jurkat cells occurred after a four-hour exposure to 25 μM H₂O₂.

In the work shown in Table 2, the indicated concentrations of hydrogen peroxide were added to the cells and they were incubated at body temperature for 30 minutes. Levels of remaining hydrogen peroxide were then measured by the xylenol-orange method and the percent of hydrogen peroxide that was removed by the various types of cells calculated in comparison to control cultures that contained no cells.

TABLE 2
Removal of Hydrogen Peroxide by the Different Cell Types

| | Hydrogen Peroxide Added to Cells (μM) | | | |
|---|---|---|---|---|
| Cell Types | 25^{a} | 50 | 100 | 200 |
| Normal PBMC | 31.7 ± 2.4^{b} | 13.8 ± 1.8 | 9.2 ± 1.9 | 11.8 ± 0.4 |
| THP-1 | 12.8 ± 0.4 | 10.4 ± 0.2 | 11.1 ± 0.0 | 13.9 ± 0.3 |
| YT-INDY | 48.0 ± 1.6 | 32.1 ± 0.0 | 30.9 ± 1.1 | 49.3 ± 2.3 |
| K-562 | 47.6 ± 1.6 | 54.8 ± 10.0 | 73.1 ± 2.1 | 73.1 ± 1.4 |
| Jurkat | 97.2 ± 6.8 | 73.0 ± 18.8 | 85.3 ± 13.0 | 96.0 ± 0.0 |

^{a}Amount of hydrogen peroxide added to the cells.
^{b}Numbers represent the percentage of hydrogen peroxide remaining in cultures for each cell type after a 30-minute exposure to different initial concentrations of hydrogen peroxide ± standard deviation. The lower the number the greater the ability of the cells to eliminate hydrogen peroxide.

The data in Table 2 indicates that normal cells and THP-1 removed more hydrogen peroxide than the other cell types. YT-INDY removed lower amounts of hydrogen peroxide, K-562 still lower amounts, and Jurkat cells removed hydrogen peroxide to the least extent.

In the work shown in Table 3, the indicated concentrations of drugs having ROS-mediated activity (cytarabine and parthenolide) were added to the cells and they were incubated at body temperature for 3 days. Viability was determined by the MTT assay. Numbers represent quadruplicate determinations and were calculated as percentages of live control cells not exposed to the drugs.

TABLE 3
Viability After Exposure to Two ROS-Utilizing Chemotherapeutic Drugs

| Cell | | | | | |
|---|---|---|---|---|---|
| Types | 3^{a} | 10 | 30 | 100 | 300 |
| | Cytarabine Added to Cells (μg/ml) | | | | |
| THP-1 | 62.3 ± 4.9 | 64.0 ± 1.9 | 56.0 ± 3.6 | 57.4 ± 2.8 | 55.3 ± 2.7 |
| YT- | 96.2 ± 4.3 | 96.5 ± 5.5 | 96.6 ± 6.4 | 79.6 ± 10.9 | 61.6 ± 7.1 |
| INDY | | | | | |
| | Parthenolide Added to Cells (μg/ml) | | | | |
| THP-1 | 99.0 ± 2.7 | 94.0 ± 1.6 | 77.4 ± 14.1 | 50.4 ± 0.4 | 47.9 ± 1.2 |
| YT- | 72.7 ± 9.6 | 59.0 ± | 79.1 ± 11.1 | 36.9 ± 0.8 | 36.2 ± 0.6 |
| INDY | | 12.0 | | | |

^{a}Amount of chemotherapeutic drug added to the cells.
^{b}Data indicates the percentage of live cells (compared to control cells without drug) remaining after drug exposure for 3-4 days ± standard deviation. The lower the number, the greater the loss of viability in response to the drug.

The data in Table 3 indicates that THP-1 cells are more susceptible to killing by cytarabine (start to die after exposure to 3 μg/ml), while YT-INDY are more easily killed by parthenolide (significant loss of viability at 10 μg/ml).

The data from these tables shows that for the 5 cell types, those that removed the greatest amount of hydrogen peroxide from the cultures generally had the least amount of negative effects from exposure to it (least growth inhibition and least loss of viability). This pattern was true for all of the tested cell types. Thus removal of hydrogen peroxide from cultures corresponds to resistance to its damaging effects. When two of the cell types were exposed to drugs having ROS-mediated activity, differences were revealed in their susceptibility to drug-mediated killing. The pattern of vulnerability to parthenolide corresponded to that seen with hydrogen peroxide, and is in synch with the present invention. The pattern of vulnerability to cytarabine needs to be further explored, and if not an artifact, might be an indication of other mechanisms taking place. For example, it is known that cytarabine is also cytotoxic because it inhibits DNA synthesis. Those skilled in the art will appreciate the manner in which the drug-susceptibility of the other cell types as well as whether other ROS-utilizing drugs act can be evaluated, to find those that act in the same differential manner as parthenolide.

### Assays:

### General Tissue Preparation

1. Obtain a 12-15 cc heparinized sample of patient's blood or a sample of solid tumor by biopsy.
2. Ship blood/tissue at ambient temperature by overnight mail to the testing site.
3. Isolate cancer cells for leukemias: purify total leukocytes using hypotonic lysis with commercially available lysing solution. Purify leukemia cells by a method best suited to the type of leukemia involved (most likely by differential centrifugation). For solid tumors, digest with proteases to obtain a single cell suspension.

### Degradation of Hydrogen Peroxide

1. Incubate single cell suspension obtained above (concentration=2×10⁵ cells/ml) at 37° C., for 30 minutes in the presence of five 2-fold dilutions of hydrogen peroxide (12.5 to 200 μM) or without hydrogen peroxide (control).
2. For test samples, remove an aliquot of 40 μl of culture supernatant from each of the 6 tubes and add directly to 400 μl of assay solution (250 μM ferrous II ammonium sulfate+25 mM H₂SO₄+100 mM sorbitol+125 μM xylenol-orange in distilled or deionized water in plastic-ware). In parallel, run a standard curve (eight 2-fold dilutions of hydrogen peroxide 3 to 400 μM hydrogen peroxide) in the same manner as test samples.
3. Incubate≧20 min at room temp. Put 90 μl of each test sample (in triplicate) or standard curve dilution into wells of a 96-well plate.
4. Read in a plate-reading spectrophotometer* at 595 nm to obtain optical density.
5. Calculate the amount of H₂O₂ remaining in the test samples by linear regression analysis. Compare the hydrogen peroxide elimination rate of the patient's cells to values established for a variety of leukemic cells with determined susceptibilities to hydrogen peroxide.

### Measurement of Superoxide Anion

1. Place 2×105 cells/well in a 96-well plate. Incubate with 100 ml/well of a 160 mM cytochrome c in phenol red-free Hanks' solution. For the blank control, incubate cells with cytochrome c containing 200 units/ml of polyethylene glycol coupled to bovine erythrocyte superoxide dismutase (PEG-SOD). To minimize the peroxide effect on cytochrome c oxidation, include 300 units/ml of polyethyethylene glycol coupled to bovine catalase (PEG-Catalase) in the cell culture. For a positive control, incubate cells with 500 ng/ml of PMA.
2. Incubate cells in a 37° C. incubator for 1 h.
3. Read the optical density (OD) using a palte reading spectrophotometer* set at 550 nm.
4. Convert OD values converted to nm superoxide based on the extinction coefficient of cytochrome c.
   If no plate reading spectrophotometer is available, samples may be placed into cuvettes and read in a regular spectrophotometer.

1. A method for determining the in vivo efficacy of drugs in the course of chemotherapy, the method comprising the steps of:
a) identifying a patient having a condition potentially susceptible to one or more candidate drugs having a particular in vivo biochemical activity;
b) providing an in vitro biochemical assay corresponding to the biochemical activity;
c) obtaining a suitable sample from the patient;
d) performing the biochemical assay on the tissue sample under conditions suitable to determine the responsiveness of the tissue sample to the biochemical activity,
e) determining a course of chemotherapy based, at least in part, on the responsiveness of the tissue sample to the biochemical activity.

2. A method according to claim 1 wherein the in vivo biochemical activity of the candidate drugs comprises the direct or indirect generation of either reactive oxygen species and/or reactive nitrogen species.

3. A method according to claim 2 wherein the in vitro biochemical assay comprises means for exposing a patient sample to reactive oxygen species and/or reactive nitrogen species; and

4. A method according to claim 3 wherein the responsiveness of the tissue sample to the biochemical activity comprises the ability of the tissue sample to inactivate the corresponding reactive species.

5. A method according to claim 1, wherein the condition is leukemia.

6. A method according to claim 5, wherein the candidate drugs are those that function by means of the generation of reactive oxygen species or reactive nitrogen species.

7. A method of identifying a compound that inhibits cancer cells in a patient, the method comprising a) providing a patient sample, b) contacting the sample with an ROS or RNS species, at one or more concentrations, and c) determining the manner or extent to which the sample is able to degrade hydrogen peroxide, d) based on the degradation of hydrogen peroxide, determine a course of drug therapy accordingly.

8. A method according to claim 7 wherein the cancer cells are leukemia cancer cells and the drugs comprise those that act through the generation of ROS species.

9. A method according to claim 8 wherein the drugs are selected from the group consisting of daunorubicin, doxorubicin, cytarabine, parthenolide, mitomycin C, adriamycin, carminomycin, 7-0-methylnogalarol, aclacinomycin A, benzanthraquinones, streptonigrin, and cisplatin.

10. A method according to claim 9 wherein the ROS species comprises hydrogen peroxide provided at a plurality of doses.

11. A system for determining the in vivo efficacy of drugs in the course of chemotherapy, for use in prescribing drugs to a patient having a condition potentially susceptible to one or more candidate drugs having a particular in vivo biochemical activity, the system comprising an in vitro biochemical assay corresponding to the biochemical activity, and wherein the assay is adapted to be performed with a suitable tissue sample from the patient under conditions suitable to determine the responsiveness of the tissue sample to the biochemical activity, the results of which are adapted to be used for determining a course of chemotherapy based, at least in part, on the responsiveness of the tissue sample to the biochemical activity.
